(19) United States
(12) Patent Application Publication
(10) Pub. No.: US 2006/0269567 A1
(43) Pub. Date: Nov. 30, 2006

(54) Universal detoxifying composition

(76) Inventor: Liu Yuen, San Gabriel, CA (US)

Correspondence Address: Paul R. Martin, 2291 Bennington Drive, Vallejo, CA, 94591, US

(21) Appl. No.: 11/433,813

(22) Filed: May 12, 2006

Related U.S. Application Data
(60) Provisional application No. 60/681,238, filed on May 16, 2005.

(51) Int. Cl. A61K 36/06; A61K 36/282; A61K 36/53; A61K 36/328; A61K 36/48; A61K 36/28; A61K 36/87; A61K 36/23; A61K 36/32; A61K 36/634; A61K 36/86

(52) U.S. Cl. 424/195.15; 424/740; 424/195.18; 424/748; 424/757; 424/764; 424/773; 424/776; 424/766; 424/745

(57) ABSTRACT

Described is a universal detoxifying composition comprising: from about 10 to about 20% by weight Radix Rehmannica, from about 8 to about 18% by weight Scutellariae Racis, about 2% by weight Rhei Rhizoma, about 1% by weight Mirabilite Pawde, from about 3 to about 13% by weight Herba Taraxaci, from about 3 to about 13% by weight Herba Violae, from about 5 to about 15% by weight Puemariae Radix, from about 1 to about 11% by weight Fructus Viticis, from about 2 to about 12% by weight Fructus Forsythiae, from about 2 to about 12% by weight Gardeniae Fructus, about 3% by weight Sophara Flavescens Aiton, about 1% by weight Gummi Olibanum, about 1% by weight Myrrha, from about 6 to about 14% by weight Cordyceps, and from about 4 to about 12% by weight Young Ji. Also disclosed is a method of treating cancer which comprises administering a therapeutically efficient amount of the composition to a patient.

### TECHNICAL FIELD

This invention relates to a universal detoxifying composition for use in the human body. The detoxifying composition is based on principles used in oriental medicine, utilizing herbs and other compounds. It can be formulated as a tonic water or pill.

### BACKGROUND OF THE INVENTION

Chinese medicine is an ancient art, having been in existence for over 4,000 years. Chinese medicine is based on different principles than are used in standard Western medicine, and utilizes natural herbs in many of its medicines in order to counter the ills of the body.

Herbs are plant, mineral, and animal substances that the body assimilates through its digestive, respiratory, and cutaneous tissues. They reorganize the body constituents recognized in Chinese Medicine (Qi, Moisture, and Blood) within Organ Networks and oust the Adverse Climates that are principles of Chinese Medicine (Wind, Heat, Cold, Dryness, and Dampness). Four basic properties—dispersing, consolidating, purging, and tonifying represent an herb's potential for achieving a particular outcome. Properties are deduced from observing the effects herbs evoke.

Herbs are used in Chinese Medicine to detoxify the body. The concepts of natural cleansing, and detoxification have been around for quite some time. In modern times, as society has increasingly been exposed to toxic compounds in the air, water, and food, it has become apparent that our ability to detoxify substances to which we are exposed is of critical importance to our overall health. Toxic substances are everywhere, . . . in the air we breathe, in the food we eat, and the water we drink. Even our bodies and the bacteria in our intestines produce toxic substances.

Toxins can damage the body in insidiuous and cumulative ways. The body has its own detoxifying system, i.e, the immune system. This system protects the body from foreign elements such as heavy metals, solvents, bacteria and viruses, for example. Once the immune system becomes overloaded, however, toxic metabolites accumulate, and we become progressively more sensitive to other chemicals, some of which are not normally toxic.

As used herein, the term “toxin” refers to any compound that has a detrimental effect on cell function or structure. Specific examples of toxins include heavy metals such as lead, mercury, cadmium, arsenic, and aluminum. Common sources of heavy metals, in addition to industrial sources, include lead from pesticide sprays, and cooking utensils, cadium and lead from cigarette smoke, mercury from dental fillings, and contaminated fish, and aluminum from antacids and cookware. Other examples of toxins include those which accumulate in the liver, such as those derived from toxic chemicals, drugs, alcohol, solvents, formaldehyde, pesticides, herbicides, and food additives.

In short, the human body is full of toxins that it's natural defense mechanisms are often unable to overcome. They accumulate in all of the organ systems of the body . . . in the liver, kidneys, blood, brain, muscles, etc.

It is known that if sufficient toxins accumulate in the body, they can frequently trigger the conversion of normally occurring cells into cancer cells. During recent years, Chinese medicine has made great progress in the treatment of tumors, i.e., cancer. Chinese medical doctors believe that the cause of cancer is due to stagnant qi, and blood stasis. It is due to the outbreak of internal heat toxins, entering of chill into the body, and cell infections. Thus, promotion of blood circulation, strengthening of body resistance to eliminate pathogenic factors, and detoxicating mechanisms must be taken to treat cancers and to strength one's body, inhibit the growth of cancer cells and regulate the internal secretion. When using the telomere enzymes of lingzhi to destroy tumor cells, it will inhibit the reproduction of tumor cells because lingzhi acids may kill tumor cells directly. In addition lingzhi contains a high content of organic germanium that can regulate the electric potential of tumor cells and damage their living environment. Using frankincense and myrrh to promote blood circulation and remove blood stasis, and using rhubarb and sodium sulphate to clear heat and promote diuresis will help also with the prevention and treatment of cancer.

The present invention is a universal herb based composition that is effective in eliminating toxins from the human body regardless of the organ system the toxin resides in, and thus effective in the prevention and treatment of cancer.

### BRIEF SUMMARY OF THE INVENTION

The present invention is a universal herbal composition effective in removing toxins from the human body. The composition is useful in removing toxins from all organ systems, but is primarily used to expel toxins from the blood, intestines, and stomach to achieve the purpose of preventing and treating cancer. The composition can be formulated as a pill, or as a tonic water.

### DETAILED DESCRIPTION OF THE INVENTION

The universal detoxifying composition of the invention can be made into a pill, or prepared as tonic water.

It has the following formulation.

(a) from about 10 to about 20% by weight Radix Rehmannica
(b) from about 8 to about 18% by weight Scutellariae Racis
(c) about 2% by weight Rhei Rhizoma.
(d) about 1% by weight Mirabilite Pawde
(e) from about 3 to about 13% by weight Herba Taraxaci
(f) from about 3 to about 13% by weight Herba Violae
(g) from about 5 to about 15% by weight Puemariae Radix
(h) from about 1 to about 11% by weight Fructus Viticis
(i) from about 2 to about 12% by weight Fructus Forsythiae
(j) from about 2 to about 12% by weight Gardeniae Fructus
(k) about 3% by weight Sophara Flavescens Aiton
(l) about 1% by weight Gummi Olibanum
(m) about 1% by weight Myrrha
(n) from about 6 to about 14% by weight Cordyceps, and
(o) from about 4 to about 12% by weight Young Ji
   More preferably, the composition has the following formulation:
(a) from about 12 to about 18% by weight Radix Rehmannica
(b) from about 10 to about 16% by weight Scutellariae Racis
(c) about 2% by weight Rhei Rhizoma.
(d) about 1% by weight Mirabilite Pawde
(e) from about 6 to about 10% by weight Herba Taraxaci
(f) from about 5 to about 10% by weight Herba Violae
(g) from about 8 to about 12% by weight Puemariae Radix
(h) from about 4 to about 8% by weight Fructus Viticis
(i) from about 5 to about 9% by weight Fructus Forsythiae
(j) from about 5 to about 9% by weight Gardeniae Fructus
(k) about 3% by weight Sophara Flavescens Aiton
(l) about 1% by weight Gummi Olibanum
(m) about 1% by weight Myrrha
(n) from about 8 to about 12% by weight Cordyceps, and
(o) from about 6 to about 110% by weight Young Ji
   Most preferably, the composition has the following formulation:
(a) about 15% by weight Radix Rehmannica
(b) about 13% by weight Scutellariae Racis
(c) about 2% by weight Rhei Rhizoma.
(d) about 1% by weight Mirabilite Pawde
(e) about 8% by weight Herba Taraxaci
(f) about 8% by weight Herba Violae
(g) about 10% by weight Puemariae Radix
(h) about 6% by weight Fructus Viticis
(i) about 7% by weight Fructus Forsythiae
(j) about 7% by weight Gardeniae Fructus
(k) about 3% by weight Sophara Flavescens Aiton
(l) about 1% by weight Gummi Olibanum
(m) about 1% by weight Myrrha
(n) about 10% by weight Cordyceps, and
(o) about 8% by weight Young Ji

The composition of this invention can be formulated either as a pill, or a tonic, that is, a water solution juice) of the ingredients of the composition. These are prepared in the following manner.

Tonic Water: The medicinal materials are cleaned, and air or sun dried. Then, the individual ingredients are ground at a high speed in asepsis environment until they become powder of 100 mesh in fineness. The mass of powder is placed in a container, and the juice extracted using 100 C water. The juice is then filtrated, and placed in a bottle. The recommended dosage is 10 cc after every meal.

Pill: The medicinal materials are cleaned, then sun or air dried. Then, the mass is ground at high speed in asepsis environment until they become powder of 120 mesh in fineness. The powder is then disinfected with infrared rays, and placed in capsules to become pills. Each pill is 400 mg. It is recommended that two pills are taken with water after every meal.

Examples of Efficacy.

Mrs. Jones, from Malaysia, age 62, was discovered to suffer from pancreatic cancer. Two hospitals confirmed that she could neither receive chemo-therapy or surgery, and that she had only three months to live. After drinking the “Detoxicating Tonic Water” of the invention for 30 days, the cancer was controlled and no longer proliferated. In fact, it was reduced in size. Originally, the patient had been lying in bed and could not get up. However, after treatment with the “Detoxicating Tonic Water” of the invention, she was able to walk for an hour everyday. After drinking the water for 10 months, her condition continued to improve.

Mr. Smith, from Los Angeles, USA, age 56, cut his lip while shaving. The wound did not heal for over a year, and it often bled. The patient consulted a doctor, and he was found to have skin cancer. He made an appointment to have chemo-therapy a month later. However, during the time he was waiting for the chemo-therapy, he drank the “Detoxicating Tonic Water” of the invention. Thereafter, he went to the Doctor's for chemo-therapy. The doctor found that his condition had improved and no chemo-therapy was needed. A year passed, and the wound coalesced. Originally the wound was ulcerated, but now he is able to grow a beard.

Ms. Lee, from San Francisco, age 26, had tumors on the inside of her legs. They were ulcerated from left to right, and from right to left. She had them removed by her doctor several times, but they always grew back. Even when she traveled, she had to bring hot water bottles to apply them to the affected area. It was very painful for her. Later, after she drank the “Detoxicating Tonic Water” for fifteen days, the tumors were gone and they did not re-occur. She has been tumor free for three years.

From the results of the clinical trials above, it can be seen that the universal detoxifying composition of the invention is effective in the treatment of cancer of various types.

Although this invention has been described in its preferred forms with a certain degree of particularity, is will be appreciated by those skilled in the art that the present disclosure of the preferred forms has been made only by way of example and that various changes in the percentage and specificity of ingredients in the formulations can be made without departing from the scope and spirit of the invention.

1. A universal detoxifying composition comprising:
(a) from about 10 to about 20% by weight Radix Rehmannica
(b) from about 8 to about 18% by weight Scutellariae Racis
(c) about 2% by weight Rhei Rhizoma.
(d) about 1% by weight Mirabilite Pawde
(e) from about 3 to about 13% by weight Herba Taraxaci
(f) from about 3 to about 13% by weight Herba Violae
(g) from about 5 to about 15% by weight Puemariae Radix
(h) from about 1 to about 11% by weight Fructus Viticis
(i) from about 2 to about 12% by weight Fructus Forsythiae
(l) from about 2 to about 12% by weight Gardeniae Fructus
(k) about 3% by weight Sophara Flavescens Aiton
(l) about 1% by weight Gummi Olibanum
(m) about 1% by weight Myrrha
(n) from about 6 to about 14% by weight Cordyceps, and
(o) from about 4 to about 12% by weight Young Ji

2. A universal detoxifying composition comprising:
(a) from about 12 to about 18% by weight Radix Rehmannica
(b) from about 10 to about 16% by weight Scutellariae Racis
(c) about 2% by weight Rhei Rhizoma.
(d) about 1% by weight Mirabilite Pawde
(e) from about 6 to about 10% by weight Herba Taraxaci
(f) from about 5 to about 10% by weight Herba Violae
(g) from about 8 to about 12% by weight Puemariae Radix
(h) from about 4 to about 8% by weight Fructus Viticis
(i) from about 5 to about 9% by weight Fructus Forsythiae
(l) from about 5 to about 9% by weight Gardeniae Fructus
(k) about 3% by weight Sophara Flavescens Aiton
(l) about 1% by weight Gummi Olibanum
(m) about 1% by weight Myrrha
(n) from about 8 to about 12% by weight Cordyceps, and
(o) from about 6 to about 110% by weight Young Ji

3. A universal detoxifying composition comprising:
(a) about 15% by weight Radix Rehmannica
(b) about 13% by weight Scutellariae Racis
(c) about 2% by weight Rhei Rhizoma.
(d) about 1% by weight Mirabilite Pawde
(e) about 8% by weight Herba Taraxaci
(f) about 8% by weight Herba Violae
(g) about 10% by weight Puemariae Radix
(h) about 6% by weight Fructus Viticis
(i) about 7% by weight Fructus Forsythiae
(j) about 7% by weight Gardeniae Fructus
(k) about 3% by weight Sophara Flavescens Aiton
(l) about 1% by weight Gummi Olibanum
(m) about 1% by weight Myrrha
(n) about 10% by weight Cordyceps, and
(o) about 8% by weight Young Ji

4. A tonic water comprising a water solution of the herbal composition of claim 1.

5. A tonic water comprising a water solution of the herbal composition of claim 2.

6. A tonic water comprising a water solution of the herbal composition of claim 3.

7. The composition of claim 1 in a pill form.

8. The composition of claim 2 in a pill form.

9. The composition of claim 3 in a pill form.

10. A method of preventing or treating cancer comprising administering a therapeutically effective amount of the herbal composition of claims 1, 2, or 3.

11. A method of preventing or treating cancer comprising administering a therapeutically effective amount of the tonic water of claims 4, 5, or 6.

12. A method of preventing or treating cancer comprising administering a therapeutically effective amount of the composition of claims 7, 8, or 9.
